(19) United States
(12) Patent Application Publication
(10) Pub. No.: US 2016/0096782 A1
(43) Pub. Date: Apr. 7, 2016

(54) METHODS AND APPARATUSES FOR PROCESSING BIO-DERIVED NORMAL NONANE

(71) Applicant: UOP LLC, Des Plaines, IL (US)

(72) Inventor: Stanley Joseph Frey, Palatine, IL (US)

(21) Appl. No.: 14/851,167

(22) Filed: Sep. 11, 2015

Related U.S. Application Data
(60) Provisional application No. 62/058,559, filed on Oct. 1, 2014.

(51) Int. Cl. C07C 5/27; C10L 10/10; C10L 1/04

(52) U.S. Cl. CPC C07C 5/2791; C10L 1/04; C10L 10/10; C10L 2200/0469; C07C 2523/40; C07C 2521/12

(57) ABSTRACT

Methods for forming bio-derived fuel products, upgrading bio-derived feedstocks, and processing bio-derived normal nonane are provided. In an embodiment, a method for forming a bio-derived fuel product includes providing a bio-derived hydrocarbon stream comprising at least about 50 wt % normal nonane and having a research octane number of less than about 10. The method further includes isomerizing the bio-derived hydrocarbon stream over a non-zeolitic, non-sulfated and/or non-halogenated catalyst to form the bio-derived fuel product with a research octane number of greater than about 50.

### CROSS-REFERENCE TO RELATED APPLICATION

This application claims priority from Provisional Application No. 62/058,559 filed Oct. 1, 2014, the contents of which are hereby incorporated by reference in its entirety.

### TECHNICAL FIELD

The technical field generally relates to methods and apparatuses for processing bio-derived normal nonane. More particularly, the technical field relates to methods and apparatuses for forming bio-derived fuel products from bio-derived normal nonane.

### BACKGROUND

As the demand for fuel increases worldwide there is increasing interest in sources other than petroleum crude oil for producing the fuel. Specifically, biological sources are being investigated for use in supplementing or replacing petroleum crude oil as the primary feedstock in hydrocarbon processing. Bio-derived sources include biomass, such as plant oils such as corn, rapeseed, canola, soybean and algal oils; animal fats such as tallow, fish oils and various waste streams such as yellow and brown greases; and sewage sludge. Bio-derived sources also include carbon-based products formed by engineered organisms, such as engineered algae cells.

Many methods have been suggested for utilizing bio-derived fuel, i.e., fuel processed from biological sources, for energy production in order to compensate for at least a portion of the fossil fuel currently used in such energy production, and thereby also decrease net CO₂ emissions in the overall energy production cycle.

Unfortunately, non-uniformity in the raw material (i.e., biomass), differences in its quality, and other similar hard-to-control variations, may cause problems in energy production cycles that rely heavily on bio-derived fuel processed from biomass. Carbon-based products formed by engineered organisms are provided with more uniformity than biomass sources. However, generally all bio-derived sources are considered to be low energy fuels, and not easily utilized for energy production. The low energy content of bio-derived sources often renders them generally inadequate for high-efficiency production of energy, such as high-temperature, high-pressure steam or electricity production.

For example, normal nonane (C₉H₂₀) may be provided as a product from an engineered organism. While a product stream of normal nonane may be highly uniform, it has little chemical application. Further, normal nonane is in the light boiling range for diesel application at about 150° C. The research octane number (RON) of normal nonane is less than zero.

Accordingly, it is desirable to provide methods and apparatuses for processing bio-derived normal nonane. Further, it is desirable to provide methods and apparatuses for upgrading a bio-derived feedstock to obtain a branched-paraffin product having an increased octane number. Also, it is desirable to provide methods and apparatuses for forming bio-derived fuel products from bio-derived normal nonane. Furthermore, other desirable features and characteristics will become apparent from the subsequent detailed description and the appended claims, taken in conjunction with the accompanying drawings and this background.

### BRIEF SUMMARY

Methods and apparatuses for forming bio-derived fuel products, upgrading bio-derived feedstocks, and processing bio-derived normal nonane are provided. In an embodiment, a method for forming a bio-derived fuel product includes providing a bio-derived hydrocarbon stream comprising at least about 50 weight percent (wt %) normal nonane and having a research octane number of less than about 10. The method further includes isomerizing the bio-derived hydrocarbon stream over a non-zeolitic, non-sulfated and/or non-halogenated catalyst to form the bio-derived fuel product with a research octane number of greater than about 50.

In another embodiment, an apparatus for upgrading a bio-derived feedstock to obtain a branched-paraffin product having an increased octane number is provided. The apparatus includes a non-zeolitic, non-sulfated or non-halogenated catalyst comprising at least one platinum-group metal component and an acidic support. Further, the apparatus includes an isomerization zone configured for contacting the bio-derived feedstock with the non-zeolitic, non-sulfated or non-halogenated catalyst at isomerization conditions to isomerize normal nonane without substantial cracking to produce the branched paraffin product with a research octane number of greater than about 50.

In another embodiment, a method for processing bio-derived normal nonane includes isomerizing the normal bio-derived nonane over a non-zeolitic, non-sulfated or non-halogenated catalyst while inhibiting cracking of the bio-derived normal nonane to form an isomerized stream with a research octane number of greater than about 50.

### BRIEF DESCRIPTION OF THE DRAWINGS

The various embodiments will hereinafter be described in conjunction with the following drawing figures, wherein like numerals denote like elements, and wherein:

FIG. 1 is a schematic diagram of an apparatus and a method for processing bio-derived hydrocarbon stream in accordance with an exemplary embodiment.

### DETAILED DESCRIPTION

The following Detailed Description is merely exemplary in nature and is not intended to limit the methods described herein. Furthermore, there is no intention to be bound by any theory presented in the preceding Background or the following Detailed Description.

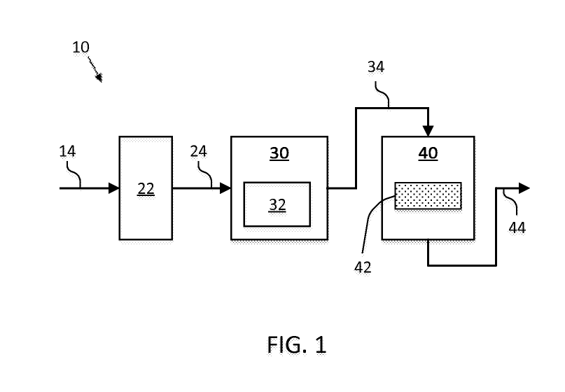

Various embodiments contemplated herein relate to methods for processing bio-derived normal nonane. FIG. 1 illustrates an exemplary apparatus 10 utilizing an exemplary method for processing a bio-derived hydrocarbon stream. In embodiments, the bio-derived hydrocarbon stream may be a bio-derived feedstock 14 or, alternatively, may be a purified stream 34 that is obtained from the bio-derived feedstock 14. As used herein, bio-derived feedstocks are those derived from plant or algae matter, and are often referred to as renewable feedstocks. Bio-derived feedstocks are not based on petroleum or other fossil fuels. In certain embodiments, the bio-derived feedstock 14 includes normal nonane. For example, the bio-derived feedstock 14 may include from about 50 weight percent (50 wt %) to about 100 wt % normal nonane. In an exemplary embodiment, the bio-derived feedstock 14 includes at least about 60 wt % normal nonane, such as at least about 70 wt % normal nonane, for example at least about 80 wt % normal nonane or at least about 90 wt % normal nonane. The exemplary bio-derived feedstock 14 has a research octane number of less than about 10, such as less than about 5, for example less than about 0. Such a bio-derived feedstock 14 may be obtained as a carbon-based product formed by engineered organisms, such as by engineered algae cells. In an exemplary embodiment, the bio-derived feedstock contains no oxygen, as produced by the engineered organism.

The bio-derived feedstock 14 may include water, nitrogen and sulfur. In FIG. 1, the bio-derived feedstock 14 may be fed to a drying unit 22. The drying unit 22 dehydrates the bio-derived feedstock 14 and removes at least a portion of, or substantially all the water, from the bio-derived feedstock 14 to form a dried bio-derived feedstock 24. An exemplary drying unit 22 includes a molecular sieve at dehydration conditions effective to selectively remove water from the bio-derived feedstock 14.

The dried bio-derived feedstock 24 may thereafter be purified to remove contaminants As shown, the dried bio-derived feedstock 24 is directed to a purification system 30 to remove contaminants, such as nitrogen compounds and sulfur compounds, among others. Trace amounts of contaminants suitable for processing may remain. In one example, purification system 30 is an adsorption system. Alternatively or additionally, a selective aromatic removal unit 32, available from UOP LLC, may be employed as part of purification system 30. After purification, a purified stream 34 is removed from the purification system 30.

In an exemplary embodiment, the purified stream 34 is a clean hydrocarbon product that is suitable for further treatment using a catalytic reaction. Specifically, the exemplary hydrocarbon product includes less than 1 ppm sulfur and less than 0.1 ppm nitrogen. An exemplary purified stream 34 includes at least about 50 wt % normal nonane, such as at least about 60 wt %, at least about 70 wt %, at least about 80 wt %, or at least about 90 wt % normal nonane. An exemplary purified stream 34 has a research octane number of less than about 10, such as less than about 5, for example less than about 0. In an exemplary embodiment, the purified stream 34 includes at least about 80 wt % normal nonane and has a research octane number of less than about 10. In another embodiment, the purified stream 34 includes at least about 90 wt % normal nonane and has a research octane number of less than about 5. Further, the exemplary purified stream 34 has a sulfur content of less than about 1 part per million (ppm) sulfur. Also, the exemplary purified stream 34 has a nitrogen content of less than about 0.1 ppm.

In various embodiments and as shown in FIG. 1, the purified stream 34 that is derived from the bio-derived feedstock 14 is a bio-derived hydrocarbon stream that is subject to isomerization to produce a branched paraffin product stream 44. However, in alternative embodiments and although not shown, it is to be appreciated that the bio-derived feedstock may be the bio-derived hydrocarbon stream that is subject to isomerization. In FIG. 1, the purified stream 34 is fed to an isomerization zone 40. In the isomerization zone 40, the purified stream 34 is contacted with an isomerization catalyst 42 to isomerize the normal nonane without substantial cracking to produce a branched paraffin product stream 44, or isomerized stream.

An exemplary isomerization catalyst 42 may be a non-zeolitic, non-sulfated and/or non-halogenated catalyst. In embodiments, the isomerization catalyst 42 has mild to medium acid strength. An exemplary isomerization catalyst 42 has a hydrogenation function. Further, an exemplary isomerization catalyst 42 comprises a supported platinum-group metal component. For example, the isomerization catalyst 42 may be a non-zeolitic, non-sulfated and/or non-halogenated catalyst comprising at least one platinum-group metal component and an acidic support. In an embodiment, the isomerization catalyst 42 includes silicon oxide and aluminum oxide. An exemplary isomerization catalyst 42 includes silicon oxide, aluminum oxide, and platinum. In an exemplary embodiment, the isomerization catalyst 42 comprises less than about 95 wt % silicon oxide, less than about 60 wt % aluminum oxide, and/or less than about 5 wt % platinum.

In an exemplary embodiment, the purified stream 34 is isomerized without substantial cracking, which may be accomplished based upon use of the aforementioned isomerization catalyst 42 and/or reaction conditions in the isomerization zone 40. As used herein, “substantial cracking” refers to the an amount of cracking sufficient to form the branched paraffin product stream 44 with a C_{5—} content of more than about 10 wt %. For example, in embodiments the isomerization zone 40 is operated at isomerization conditions including, independently, a pressure of from about 3.4 barg (about 50 psig) to about 48 barg (about 700 psig), a molar hydrogen-to-hydrocarbon ratio of from about 0.1 to 10, a liquid hourly space velocity of from about 0.2 to about 10 hr⁻¹, and a temperature of from about 150° C. to about 400° C. In an exemplary embodiment, the isomerization zone 40 is configured as a fixed-bed catalytic reactor.

In an exemplary embodiment, the branched paraffin product stream 44 is produced with a research octane number of greater than about 50. An exemplary isomerization zone 40 forms the branched paraffin product stream 44 with a research octane number of greater than about 75 based upon the aforementioned operating conditions. In an exemplary embodiment, the isomerization zone 40 operated at the aforementioned operating conditions forms the branched paraffin product stream 44 with a C_{5—} content of less than about 5 wt %.

During the isomerization process, at least about 10 wt %, such as at least about 15 wt %, for example at least about 25 wt % of the normal nonane is converted to an isononane. The exemplary branched paraffin product stream 44 may be formed with isononanes including methyloctanes, dimethylheptanes, ethylheptanes, trimethylhexanes, ethyl-methylhexanes, tetra-methylpentanes, ethyl-dimethylpentanes, ethyl-dimethylpentanes,

In an embodiment herein, apparatus 10 of FIG. 1 provides a method for upgrading the bio-derived feedstock 14 to obtain the branched paraffin product stream 44 having an increased octane number. The exemplary method includes contacting the bio-derived feedstock 14 in the isomerization zone 40 with the isomerization catalyst 42 comprising at least one platinum-group metal component and an acidic support at isomerization conditions to isomerize normal nonane without substantial cracking to produce the branched paraffin product stream 44 with a research octane number of greater than about 50.

Further, the apparatus 10 provides a method for processing bio-derived normal nonane including isomerizing the normal bio-derived nonane over a non-zeolitic, non-sulfated or non-halogenated catalyst 42 while inhibiting cracking of the bio-derived normal nonane to form an isomerized stream 44 with a research octane number of greater than about 50.

Accordingly, methods and apparatuses for processing bio-derived normal nonane have been described. The various embodiments comprise upgrading a normal nonane stream having a research octane number of less than 10 to a branched paraffin product stream having a research octane number of greater than 50. Further, the embodiments provide such a branched paraffin product stream by isomerizing the normal nonane without substantial cracking, i.e., without forming more than about 10 wt % of C_{5—} components. In exemplary embodiments, the isomerization process forms the branched paraffin product stream with less than about 5 wt % of C_{5—} components. Substantial cracking is avoided through the use of the described exemplary catalyst at the described exemplary isomerization conditions. It is noted that deoxygenation processes are avoided by the methods and apparatuses herein, as the exemplary bio-derived feedstock is free of oxygen. Further, the methods and apparatuses described herein provide for formation of branched paraffin products rather than aromatic products.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the subject matter. Rather, the foregoing detailed description will provide those skilled in the art with a convenient road map for implementing an exemplary embodiment. It being understood that various changes may be made in the function and arrangement of elements described in an exemplary embodiment without departing from the scope as set forth in the appended claims.

What is claimed is:

1. A method for forming a bio-derived fuel product, the method comprising the steps of:
providing a bio-derived hydrocarbon stream comprising at least about 50 wt % normal nonane and having a research octane number of less than about 10; and
isomerizing the bio-derived hydrocarbon stream over a non-zeolitic, non-sulfated and/or non-halogenated catalyst to form the bio-derived fuel product with a research octane number of greater than about 50.

2. The method of claim 1 wherein providing the bio-derived hydrocarbon stream comprises providing the bio-derived hydrocarbon stream comprising at least about 90 wt % normal nonane and having a research octane number of less than about 5.

3. The method of claim 1 wherein providing the bio-derived hydrocarbon stream comprises providing the bio-derived hydrocarbon stream having a research octane number of less than about 0.

4. The method of claim 1 wherein isomerizing the bio-derived hydrocarbon stream comprises forming the bio-derived fuel product with a research octane number of greater than about 75.

5. The method of claim 4 wherein providing the bio-derived hydrocarbon stream comprises providing the bio-derived hydrocarbon stream having a research octane number of less than about 0.

6. The method of claim 1 wherein isomerizing the bio-derived hydrocarbon stream forms the bio-derived fuel product with a C_{5—} content of less than about 5 wt %.

7. The method of claim 1 wherein the bio-derived hydrocarbon contains no oxygen as produced from a biological organism.

8. The method of claim 1 further comprising:
removing nitrogen from the bio-derived hydrocarbon stream to provide the bio-derived hydrocarbon stream with a nitrogen content of less than about 0.1 parts per million (ppm).

9. The method of claim 1 wherein isomerizing the bio-derived hydrocarbon stream comprises contacting the bio-derived hydrocarbon stream with the catalyst, wherein the catalyst comprises a supported platinum-group metal component, in a catalytic isomerization zone maintained at isomerization conditions comprising a pressure of from about 3.4 barg (about 50 psig) to about 48 barg (about 700 psig), a molar hydrogen-to-hydrocarbon ratio of from about 0.1 to 10, a liquid hourly space velocity of from about 0.2 to about 10 hr⁻¹ and a temperature of from about 150° C. to about 400° C.

10. The method of claim 1 wherein isomerizing the bio-derived hydrocarbon stream comprises contacting the bio-derived hydrocarbon stream with the catalyst, wherein the catalyst comprises silicon oxide, aluminum oxide, and platinum.

11. The method of claim 1 wherein isomerizing the bio-derived hydrocarbon stream comprises contacting the bio-derived hydrocarbon stream with the catalyst, wherein the catalyst comprises silicon oxide and aluminum oxide.

12. The method of claim 1 wherein isomerizing the bio-derived hydrocarbon stream comprises contacting the bio-derived hydrocarbon stream with the catalyst, wherein the catalyst comprises less than about 95 wt % silicon oxide, less than about 60 wt % aluminum oxide, and less than about 5 wt % platinum.

13. The method of claim 1 wherein isomerizing the bio-derived hydrocarbon stream comprises contacting the bio-derived hydrocarbon stream with the catalyst, wherein the catalyst has a hydrogenation function.

14. The method of claim 1 wherein isomerizing the bio-derived hydrocarbon stream over the non-zeolitic, non-sulfated or non-halogenated catalyst comprises isomerizing the bio-derived hydrocarbon stream over a non-zeolitic catalyst.

15. The method of claim 1 wherein isomerizing the bio-derived hydrocarbon stream over the non-zeolitic, non-sulfated or non-halogenated catalyst comprises isomerizing the bio-derived hydrocarbon stream over a non-sulfated catalyst.

16. The method of claim 1 wherein isomerizing the bio-derived hydrocarbon stream over the non-zeolitic, non-sulfated or non-halogenated catalyst comprises isomerizing the bio-derived hydrocarbon stream over a non-halogenated catalyst.

17. An apparatus for upgrading a bio-derived feedstock to obtain a branched-paraffin product having an increased octane number, the apparatus comprising:
a non-zeolitic, non-sulfated or non-halogenated catalyst comprising at least one platinum-group metal component and an acidic support;
an isomerization zone configured for contacting the bio-derived feedstock with the non-zeolitic, non-sulfated or non-halogenated catalyst at isomerization conditions to isomerize normal nonane without substantial cracking to produce the branched paraffin product with a research octane number of greater than about 50.

18. The apparatus of claim 17 further comprising a nitrogen removal unit configured to remove nitrogen from the bio-derived feedstock to provide the bio-derived feedstock with a nitrogen content of less than about 0.1 parts per million (ppm).

19. The apparatus of claim 17 further comprising a drying unit configured to remove water from the bio-derived feedstock.

20. A method for processing bio-derived normal nonane, the method comprising the step of:
isomerizing the normal bio-derived nonane over a non-zeolitic, non-sulfated or non-halogenated catalyst while inhibiting cracking of the bio-derived normal nonane to form an isomerized stream with a research octane number of greater than about 50.
